(19) United States
(12) Patent Application Publication
(10) Pub. No.: US 2022/0081147 A1
(43) Pub. Date: Mar. 17, 2022

(54) System and Method for Containing and Dispensing Fluids

(71) Applicant: Lameplast, S.r.l., Novi di Modena (MO) (IT)

(72) Inventors: Enrico Salvarani, Carpi (MO) (IT); Monica Foglio Stobbia, Novellara (RE (IT); Filippo Fangarezzi, San Martino di Mugnano (MO) (IT); Davide Andreetto, Este (PD) (IT); Philip Bourgeois, Perrysburg, OH (US)

(21) Appl. No.: 17/475,380

(22) Filed: Sep. 15, 2021

Related U.S. Application Data
(60) Provisional application No. 63/192,162, filed on May 24, 2021.
(60) Provisional application No. 63/078,765, filed on Sep. 15, 2020.

(51) Int. Cl. B65D 1/02; B65D 83/00; B65D 51/00; B65B 3/00; B65B 55/02; A61J 1/20; B65D 73/02; A45D 34/00; A61M 5/24

(52) U.S. Cl. CPC B65D 1/023; B65D 83/0055; B65D 51/002; B65B 3/003; B65B 55/02; A61M 5/2466; A61J 1/201; B65D 73/02; A45D 34/00; A61M 5/2425; A61J 1/2096

(57) ABSTRACT

An integral injection molded strip (10) of a single plastic material is provided, the strip comprising a plurality of vials (20) connected by frangible (breakable) connecting members (38). Each vial (as molded as part of the integral injection molded strip) has a neck portion (30) with an internal delivery passage (31) and a squeezable body portion (22), the squeezable body portion having an internal chamber (23) for holding a single dose of a fluid product (50), the chamber (23) having an open top end (24) fluidly connected to the internal delivery passage (31) in the neck portion, and the chamber having a bottom end (25) adapted for filling with the fluid product. The internal delivery passage (31) (of the neck portion) has an open bottom end (33) fluidly connecting to the body chamber, an opposing open top end (32), and an integral injection molded internal membrane (34) (formed in the delivery channel during injection molding of the integral strip of vials) that seals the delivery passage (31) prior to being pierced by a needle (62) of a dispensing device (60).

### FIELD OF THE INVENTION

The present invention relates to systems and methods for containing and dispensing fluids, including a container for fluid attachable to one or more different dispensing devices.

### BACKGROUND

There is a need in the fields of pharmaceutical, cosmetic, diagnostic, dental, veterinary, food and chemical, for example, for dispensing a fluid (liquid or other flowable product such as a gel or paste) through a dispensing aperture, typically in a specific dose (weight or volume) and typically requiring sterility of the product and its system for delivery. Prior art systems have one or more problems relating to breakage of glass components, use of different materials that require different disposal methods (e.g., not easily recyclable), contamination (bacterial or lubricants) from system components or method of use, expense of manufacture, and injury to the user (e.g., via needle injuries or glass breakage). The present invention addresses this need.

### SUMMARY OF THE INVENTION

A container for fluid is provided that is configurable for use with a variety of dispensing devices, such as a needle or syringe, the latter being attachable to the container for delivery of the fluid product

In one embodiment, an integral container (a vial) comprises a flexible plastic body having a neck portion, open at the top end, for delivery of the fluid from the container body. The neck portion includes an internal membrane, within a delivery passage in the neck that first seals the passage and after puncture of the membrane by a needle or luer (of a separate dispensing device) allows passage of the fluid from the body through the neck passage and needle or luer. An attachment member (e.g., thread or snap lock mechanism) on the neck enables attachment of the separate dispensing device.

Preferably, the integral container body and neck form a vial, for delivery of a single dose of the fluid product.

Preferably a strip of such vials is formed as an integral injection molded strip comprising a plurality of vials and frangible connecting members that temporarily connect the vials to form the integral strip, but which frangible connecting members can be broken (e.g., by bending or twisting) to release a vial from the strip.

Preferably, the strip of vials are filled with the fluid product simultaneously, in a single filling step where all vials are filled with a desired dose of the fluid product. In one embodiment, the container body (of each injection molded vial in the strip) is injection molded with an open bottom end, opposite the neck portion, and the filling step comprises simultaneous filling of all vials in the strip by introducing the fluid product into the open bottom ends. The filled containers can then each be sealed by closing (e.g., heat sealing) the bottom ends. The strip of vials can be sterilized (e.g., by Gamma or Eto sterilization) prior to filling and sealing of the container bottoms. The open neck end of each container can be temporarily closed by attaching a closure cap with a mating attachment member (e.g., thread or snap lock), to keep the membrane space sterile prior to uncapping and attaching the dispensing device (e.g., inserting a dual needle luer into the top end of the vial to pierce the membrane).

In use, a single fluid filled and capped vial is separated from the strip and is ready for use. The cap is readily removed by for example unscrewing the threaded cap, and the dispensing device, such as a needle or syringe, is attached to the neck portion of the container. The dispensing device has an attachment portion (e.g. a thread or snap on locking mechanism) that allows it to be attached to the neck portion in fluid tight engagement, and includes a needle or luer that punctures the internal membrane of the neck to allow passage of the fluid from the container body, into and through the first end of the needle or luer that punctures the membrane, and out the opposite open end of the needle or luer. The flexible container body is made of a soft plastic material (such as polyolefin, e.g. polyethylene or polypropylene, including copolymers and blends thereof) or the like of a thickness and flexibility such that dispensing can be activated by squeezing the container body with the user's finger pressure to force the fluid product from the container body and into and through the needle or luer that has punctured the membrane, and out the other open end of the needle or luer for delivery to a person or animal, or to a syringe for subsequent dispensing from the syringe.

### BRIEF DESCRIPTION OF THE FIGURES

FIG. 1 shows one embodiment according to the invention; on the left hand side is shown a single integral injection molded strip (10) comprising a plurality of single-dose vials (20) connected by frangible connecting members (38), and on the right hand side, the same strip with each of the vials now positioned to receive a separate cap (40), wherein the vials strip (10) and caps (40) can be pre-assembled (as shown by arrows) before filling.

FIG. 2 shows the plurality of vials in the strip (10) of FIG. 1 after being capped (at the top ends), filled (via the open bottom ends), and then heat sealed to close the bottom ends of the filled vials.

FIG. 3 shows a double-ended needle dispensing device for use with one of the filled vials of FIG. 2 (after the filled vial is separated from the strip by breaking the adjoining frangible connecting members), the left view being in cross section and the right view a plan view, showing the dispensing device now attached to the open top end of the delivery passage in the neck portion of the filled vial (after removing the cap), wherein such attachment of the delivery device both punctures the membrane in the internal delivery passage in the neck portion of the vial and provides an injection needle for ultimate delivery of the single dose of fluid product into a patient by (squeezing the flexible body portion of the filled vial).

FIG. 4 shows on the left hand side, an injection molded single-dose vial (120) according to a second embodiment of the invention, and on the right hand side, the same vial now positioned to receive a separate cap (140), wherein the vial and cap can be pre-assembled (as shown by arrows) before filling.

FIG. 5 shows on the left-hand side, the vial of FIG. 4 after attaching the cap (and still open at the bottom end for subsequent filling), and on the right hand side a cross section of the filled vial (after filling with the fluid product, heat sealing the bottom end, and removal of the cap from the top end), showing the internal fluid delivery passage of the neck portion and the internal membrane sealing the delivery passage.

FIG. 6 shows on the left hand side, a syringe dispensing device with a luer lock attachment portion for use with the filled vial of FIG. 5 (after removal of the cap), in the center view, the luer lock of the dispensing device now attached to the open top end of the delivery passage in the neck portion of the filled vial, wherein such attachment of the delivery device both punctures the membrane in the internal delivery passage in the neck portion of the vial and provides a syringe body (collection tube) enabling dispensing of the fluid product from the vial chamber into the syringe body (by squeezing the flexible vial body portion), and on the right hand side a cross sectional view of the luer lock attachment of the dispensing syringe and top end of the vial; the vial is then detached from the syringe body allowing the filled syringe to deliver the fluid product to a patient.

FIG. 7 shows a single integral injection molded strip (210) according to a third embodiment of the invention (prior to filling and heat sealing), the strip comprising a plurality of single-dose vials (220) connected by frangible connecting members (238) similar to the first embodiment, but the integral injection molded strip in this embodiment including an integrally injection molded removable closure (239) at the top end of each vial (which obviates the need for a separate cap as in the first embodiment of FIGS. 1-3),

FIG. 8 shows a use case for the third embodiment, the left hand side showing removal (by twisting and breaking off) the closure at the top end of the filled and heat sealed vial (after the filled vial is separated from the strip by breaking the adjoining frangible connecting members), in the center view, a separate double-ended needle dispensing device (and covering sheath) for use with the filled vial, and on the right hand side showing the dispensing device, now attached to the open top end of the delivery passage in the neck portion of the filled vial, wherein such attachment of the delivery device both punctures the membrane in the internal delivery passage in the neck portion of the vial and provides an injection needle for ultimate delivery of the single dose of fluid product into a patient by (squeezing the flexible body portion of the filled vial); the protective sheath is removed from the delivery needle after attachment to the vial (for user protection from injury by the delivery needle).

FIG. 9 shows a method according to one embodiment of the injection.

FIG. 10 shows a method according to another embodiment of the invention.

### DETAILED DESCRIPTION

### Embodiment A

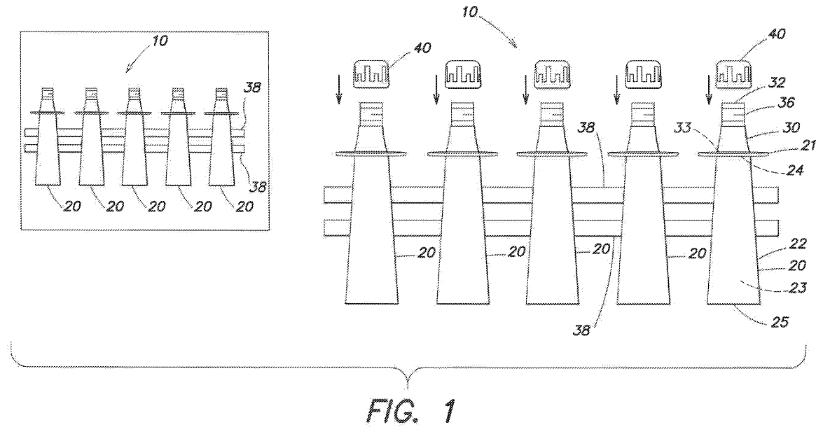

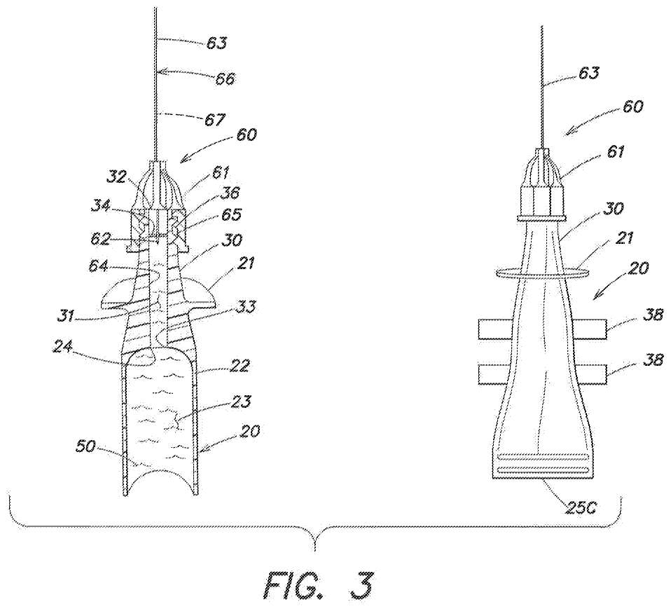

FIGS. 1-3 illustrate one embodiment of an apparatus and method of the invention. An integral injection molded strip (10) of a single plastic material is provided, the strip comprising a plurality of vials (20) connected by frangible (breakable) connecting members (38). Each vial (as molded as part of the integral injection molded strip) has a neck portion (30) with an internal delivery passage (31) and a squeezable body portion (22), the squeezable body portion having an internal chamber (23) for holding a single dose of a fluid product (50), the chamber (23) having an open top end (24) fluidly connected to the internal delivery passage (31) in the neck portion, and the chamber having a bottom end (25) adapted for filling with the fluid product. The internal delivery passage (31) (of the neck portion) has an open bottom end (33) fluidly connecting to the body chamber, an opposing open top end (32), and an integral injection molded internal membrane (34) (formed in the delivery channel during injection molding of the integral strip of vials) that seals the delivery passage (31) prior to being pierced by a needle (62) of a dispensing device (60). The vial neck portion (30) has an outer thread (36) for attaching the dispensing device (e.g., via a mating inner screw thread (65) on one end of the separate dispensing device). A separate cap (40) is provided for temporarily closing the top end of the neck portion and delivery passage, to maintain sterility of the membrane and delivery passage. A single dose of a fluid product (50) is introduced into the body chamber (23), via the open bottom end (25) of the vial (as injection molded); the open bottom end of the filled vial is then sealed, e.g. by heat sealing, to form a closed bottom end (25C) of the filled vial.

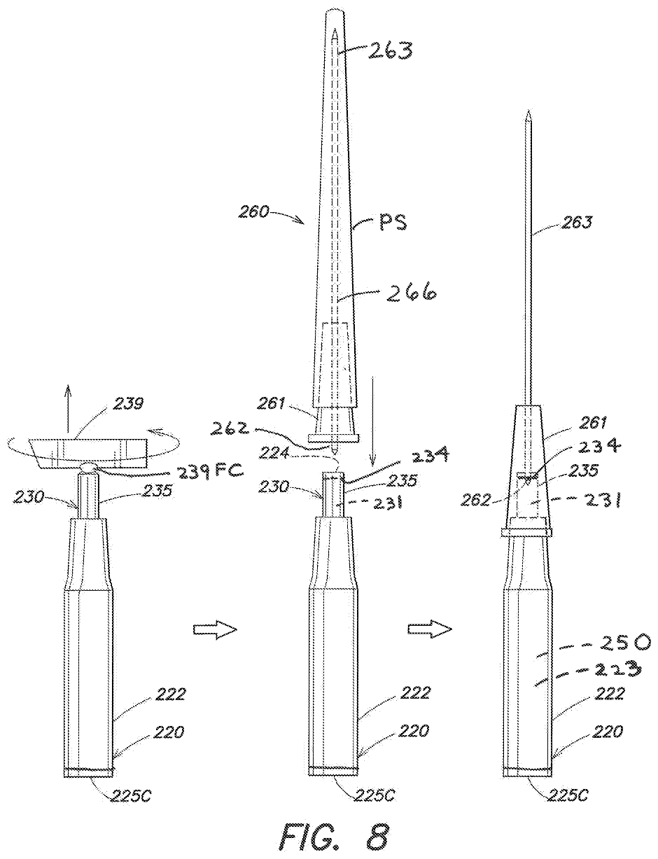

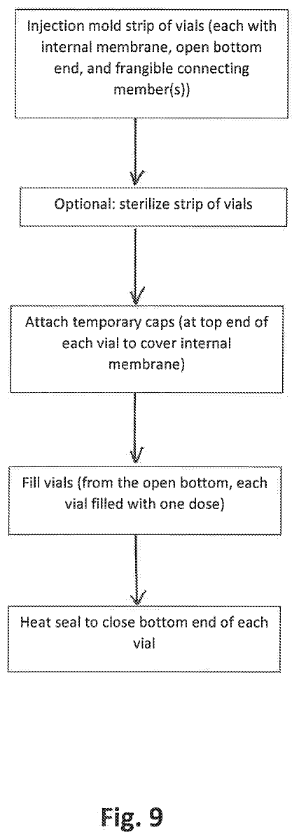

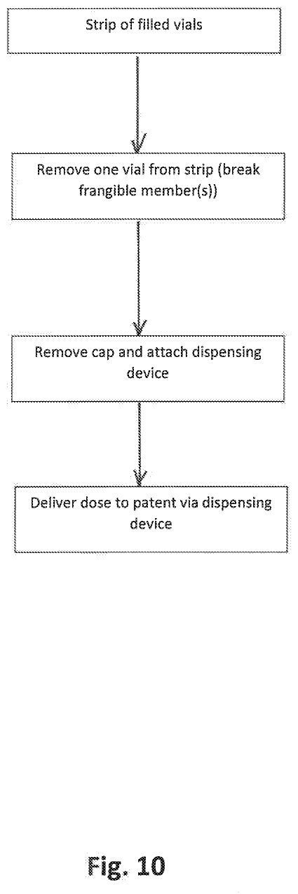

The separate delivery device 60 has a hollow cylindrical needle (66), open at both ends (63,62), with an open delivery channel (67) extending between the opposing ends (63, 62). The needle has a first patient injection end (63) (e.g., for injection of the single fluid dose from the vial to a patient) that extends from an attachment portion (61) on the other end of the delivery device; the injection needle (63) may be housed in a removable protective sheath (e.g., as shown in FIG. 8, center view), to protect the user while handling the device. The attachment portion (61), at an opposing second end of the delivery device, has an internal open aperture or bore (64) that is configured to fit over the outer surface of the vial neck portion, and includes an inner screw thread and/or snap mechanism (65) on the inner surface of the open aperture (64) for attaching to an outer thread (36) on the open top end of the vial neck portion. The second end (62) of the needle (66) extends into the open aperture (64), and protects the user from engagement with the needle end (62) prior to assembly with the vial. The second end (62) is configured to pierce the membrane (34) in the neck portion of the vial when the attachment portion is locked onto the neck portion of the vial. In use, the opposite open delivery end (63) of the needle (66) will deliver to the patient the fluid dose (50) contained in the body portion (23) of the vial, the fluid dose traveling from the inner chamber (23) of the vial body portion (22), through the delivery passage (31) in the vial neck portion (30), into the second end (62) of the needle (that has pierced the membrane (34)), through the delivery channel (67) of the needle (66) and out the opposing injection end (63) of the needle (66).

The needle (66) may be configured for injection to a person or animal (depending on the intended use). A protective sheath or cap (if provided) is temporarily positioned over the needle delivery end (63) for protecting a user from needle injury. A user unscrews the protective cap (40) of the vial (20) and opens the first end (62) of the needle (66). The user screws the attachment portion (61) onto the vial neck thereby inserting the second end (62) of the needle into the delivery channel (23) and piercing the membrane (34) of the vial neck portion. The user then uncaps the primary injection needle (63) and allows the flow of the fluid dose (50) into the patient.

In summary, an injection plastic vial (20) is linked by frangible connecting members (38) to a plurality of other vials designed to compose a strip (10), each vial (20) has a hollow body chamber (23) for containing a single dose of fluid (50). The vial may include:
A threaded neck (30) to allow the screwing of a needle (60) or other delivery device onto the neck portion (30),
A protective ring (21) extending radially outwardly beyond the outer circumference of the neck portion (30), to protect the user from the needle, and
An internal septum (34) that keeps the product (50) sealed until its perforation.
   Optionally, a cap (40) is provided that removably mates with the thread of the neck (30) to allow the vial neck area to remain clean (sterile) during filling and prior to use. The thread (36) of the neck (30) of the vial is also compatible with a commercial double end or customized needle (60).

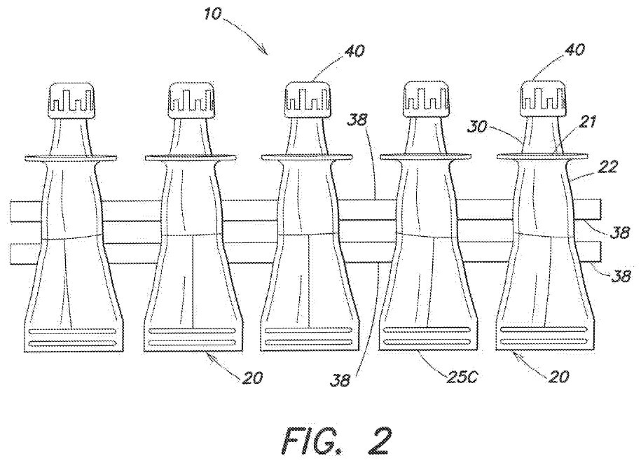

The vials (20) are manufactured by injection molding a strip of vials (e.g., between a pair of injection molds that form the outer and inner surfaces of the vials and connecting members 38), and the vials of the strip are then assembled to the caps (40) as shown in FIG. 1 (right side) and FIG. 2. The vial strip (10) and caps (40) are made separately with injection technology, and the vial strip (10) and caps (40) are pre-assembled before filling. The vial strip (10) is supplied to a filling plant, where each vial (20) is filled and heat sealed, preferably all vials being simultaneously filled in one step, and then simultaneously heat sealed and closed in one step. Prior to that, the vials (20) may be sterilized, if necessary. The final product can be sold together with commercial needles, or the final user can provide their own needle. To prepare the system for use, a user unscrews the protective cap (40) of the vial (20) and opens the first side (63) of the needle (66). The user inserts the second end (62) of the needle all the way into the vial, then uncaps the primary needle (63). The second needle (62) pierces the membrane (34) and allows the flow of product (50). The user then injects the needle (63) into the patient body and squeezes the vial body (22) to enable dispensing of the product (50).

In one embodiment, the strip (10) of plastic vials is configured for injectable preparations, like vaccines, aimed to replace current glass vials and syringes to improve safety and user experience. The neck (30) of the vial is designed to fit a commercial needle (60) for dental anesthesia. The inner membrane (34) will be pierced by the needle (62) and allow the flow of product (50). The strip (10) is made of five vials (20), all vials being filled simultaneously, and then heat sealed and closed simultaneously.

### Embodiment B

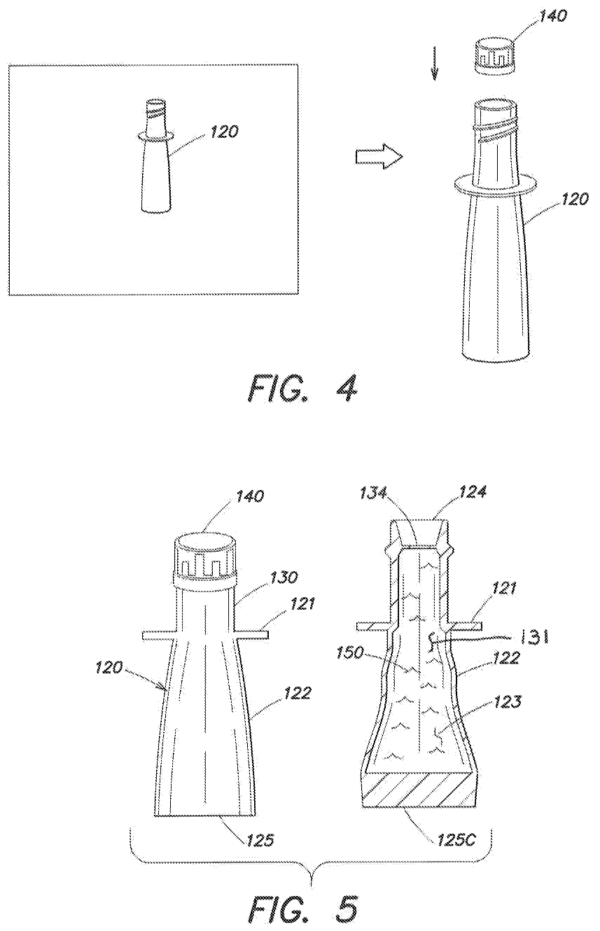

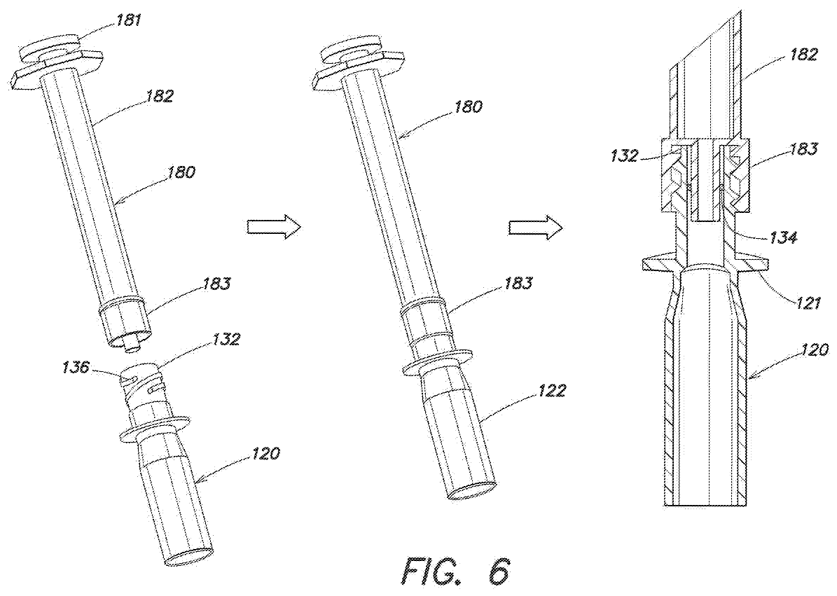

FIGS. 4-6 illustrate a second embodiment B of the invention, in which similar/corresponding elements to those of the first embodiment A of FIGS. 1-3 are labeled with a 100 series set of reference numbers (e.g., vial 20 in FIGS. 1-3, is vial 120 in FIGS. 4-6). Although not shown in FIGS. 4-6, the vials are understood to be manufactured as part of a strip of vials similar to what is described and shown in the first embodiment A. The second embodiment illustrates an alternative type of delivery device for use with the vial.

As shown in FIGS. 4-8, a plastic vial (120) is injection molded as part of a strip of vials (not shown, but similar to strip (10) of embodiment A in FIGS. 1-3) and has a squeezable hollow body portion (122) with an internal chamber (123) for containing a single dose of fluid (150). The vial includes:
A threaded neck (130) to allow the screwing of a syringe (180) or other delivery device onto the neck portion (130) of the vial; and
An internal membrane or septum (134) in the delivery passage (131) of the neck portion that keeps the product (150) sealed until the membranes perforation.

Optionally, a cap (140) is provided that mates with the thread (136) of the neck portion (130) and allows the vial neck area to remain clean. The thread (136) of the neck (130) of the vial is compatible with the neck of commercial syringes (180). The vial is manufactured by injection molding and the preassembled with caps (140) before filling. The vial strip is then supplied to the filling plant, where all vials (120) of the strip are simultaneously filled, and simultaneously heat sealed. Prior to filling and heat sealing, the vials (120) may be sterilized as necessary. In use, a user screws the syringe male luer (183) end into the vial open top end (132). The syringe made luer 1(83) breaks the inner membrane (134) and allows the flow of the fluid product (150). By moving back the syringe plunger (181) the fluid dose (150) is withdrawn from the vial (120) and goes into the syringe body (182). The syringe can then be connected with a needle to inject the fluid into the patient.

### Embodiment C

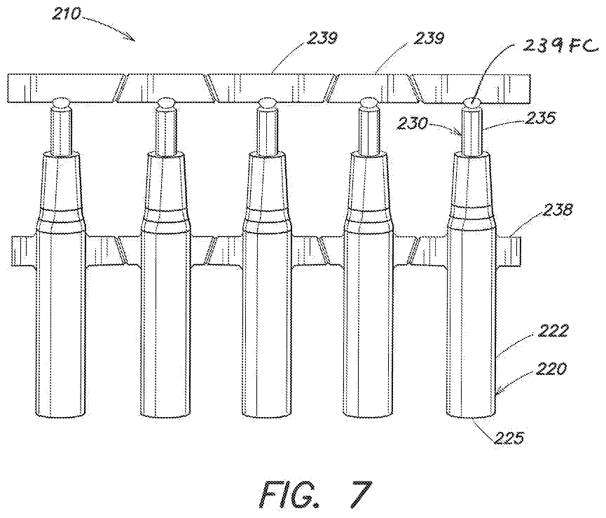

FIGS. 7-8 illustrate a third embodiment C of the invention, in which similar/corresponding elements to those in the first embodiment A of FIGS. 1-3 are labeled with a 200 series set of reference numbers (e.g., vail 20 in FIGS. 1-3 is vial 220 in FIGS. 7-8).

The vials (220) are manufactured as part of a strip (210) of vials, similar to what is described and shown in the first embodiment A. The third embodiment illustrates an alternative type of temporary closure or cap for use with the vial. The unit-dose vials (220) of the strip each have a male luer fitting (235) for connection with a needle device (260) or other delivery device.

Each plastic vial (220) is linked to a plurality of other vials in the form of an integral injection molded strip (210) connected by frangible connecting members (238), each vial (220) having a neck portion (230) with a delivery passage (231) and internal membrane (234), the delivery passage being connected to a hollow body portion (222) having an internal chamber (223) for containing a single dose of fluid (250). The vial (220) includes:
A male luer neck (235) on the neck configured to allow the attachment of a needle (260) or other delivery device; and
A temporary closure element (239) for each vial (220) that is injection molded as a single body (integral) with the vials (220) and strip (210); the closure or cap has a frangible connecting portion (239FC) to the neck portion that can be broken by the user after filling and prior to attachment of the delivery device.

The vials (220) are manufactured by injection molding technology (in a mold at elevated temperature and pressure). The vial strip (210) is then supplied to the filling plant, where all vials (220) of the strip (210) are simultaneously filled and then simultaneously heat sealed. Prior to filling and sealing, the vials may be sterilized if necessary. The final product can be sold together with commercial needles, or the final user procure the needle (e.g. a delivery device (260) having a double ended needle (266), hub (261) and needle protective sheath (PS)).

In use, the user opens the vial (220) by breaking off the frangible closure (239), inserts the second needle end (262) into the now open top end (224) of the vial to pierce through the membrane (234), and then uncaps the opposing first injection needle end (263) by removing the protective sheath (PS). Then the user injects the first needle end (263) into the patient body and squeezes the vial body portion (222) to dispense the fluid dose (250) into the patient.

### Various Embodiments

Single dose vials are provided according to the invention, made by injection molding, that can be assembled to a separate delivery device, to be used to dispense fluid (flowable) substances by squeezing the vial itself.

The single use vial, to be used in connection with a needle delivery device, may include a protection mechanism against needle injuries (e.g., protruding radial ring (21) and/or bore (64)).

The above described technology can be useful for different production volumes, i.e., number of vials produced and size of fluid dose. In other embodiments, the container can be connected with another device such as a bag, connector, applicator or the like, to be used in different applications in the fields of pharmaceutical, cosmetic, dental, veterinary, food and chemical fluid products.

By manufacturing the vials as an integral strip of multiple vials, the time, cost and equipment costs are substantially reduced. In addition, the time, cost and equipment costs for filling and heat sealing are substantially reduced.

The disposal of the vials can be made in a sustainable way, with the entire strip of vials made of a single recyclable material. The additional components (cap and delivery device) can be easily disassembled, and thus need not be part of a single recycle material stream

The filling can be done at room temperature, allowing the filling of heat-sensitive products.

Once filled, the vial neck portion remains hermetically closed until its use.

The vial has the superior quality of an injection molded article (no flashes, no sharp edges, repeatable dimensions, excellent surface finish).

This solution is user friendly, requiring a lower number of steps compared to other solutions and the steps being easy to understand and to perform by a non-professional user.

The injection can also be auto-administered (by the patient).

Being composed of a lower number of components, and produced with a cost effective injection molding technology, the cost of the device is lower than other options.

1. An integral injection molded strip made of a single plastic material and comprising a plurality of single-use vials, each vial adapted to be assembled to a needle or another dispensing device for delivery of a single dose of a fluid product from the vial by squeezing the vial itself,
each single-use vial comprising an injection molded integral neck portion and a flexible body portion of the single plastic material, the flexible body portion having an internal chamber for holding a single dose of the fluid product and flexible body wall adapted for squeezing the fluid product out an open top end of the chamber into an open delivery passage in the neck portion, the delivery passage having an open bottom end in fluid communication with the internal chamber for receiving the fluid product and an opposing open top end for delivery of the fluid product out the top end of the vial,
an injection molded internal membrane within the delivery passage that seals the delivery passage and, after puncture of the membrane by a needle or luer fitting of a separate dispensing device allows passage of the fluid product from the body chamber through the delivery passage and out the open top end,
an attachment member on the neck portion that enables attachment of the separate dispensing device,
the flexible body portion having an injection molded open bottom end, opposite the neck portion, adapted for simultaneously filling of all vials of the strip, each with the single dose of the fluid product, in a single filling step by introducing the fluid product into the open bottom ends.

2. The strip of claim 1, wherein
the strip of vials including injection molded frangible connecting members that temporarily connect the vials to form the integral strip, the frangible connecting members adapted to be broken by bending or twisting to release each vial from the strip.

3. A strip of filled and sealed vials comprising the strip of claim 1, wherein all vials are simultaneously filled with a single dose by introducing the fluid product into the open bottom ends and the open bottom ends are then sealed by simultaneously heat sealing the bottom ends.

4. The strip of claim 1, wherein
each vial includes a projecting portion on the outer surface of the neck portion for protection against needle injuries.

5. The strip of claim 1, wherein the vials are adapted for applications in the fields of pharmaceutical, cosmetic, dental, veterinary, food and chemical fluid products.

6. A method of making a strip of vials according to claim 1, wherein the strip of vials is formed as an integral injection molded strip comprising the plurality of vials and frangible connecting members that temporarily connect the vials to form the integral strip, the frangible connecting members adapted to be broken by bending or twisting to release each vial from the strip.

7. The method of claim 6, wherein the plurality of vials of the strip are filled with the fluid product simultaneously in a single filling step where all vials are filled with a single dose of the fluid product.

8. The method of claim 6, wherein the body portion of each injection molded vial in the strip is injection molded with an open bottom end, opposite the neck portion, and the filling step comprises simultaneous filling of all vials in the strip by introducing a single dose of the fluid product into the open bottom ends, and the filled vials are then heat sealed by simultaneously closing the bottom ends of all vials.

9. The method of claim 8, wherein the strip of vials is sterilized by Gamma or Eto sterilization prior to filling and sealing the bottom ends of the vials.

10. The method of claim 6, wherein the open neck end of each vial is temporarily closed by attaching a closure cap with a mating attachment member to keep the membrane and delivery passage sterile prior to uncapping and attaching the dispensing device.

11. A method of using the strip of claim 1, wherein a single fluid filled vial is separated from the strip and a dispensing device comprising a needle or syringe is attached to the neck portion of the container.

12. The strip of claim 1, wherein the dispensing device has an attachment portion comprising a thread or snap on locking mechanism adapted to be attached to the neck portion in fluid tight engagement, and includes a needle or luer that punctures the internal membrane of the neck portion to allow passage of the fluid product from the body portion, into and through the first end of the needle that punctures the membrane, and out the opposite open end of the needle or luer.

13. The strip of claim 1, wherein the single plastic material is a soft plastic material comprising a polyolefin, polyethylene and/or polypropylene, including copolymers and blends thereof, of a thickness and flexibility such that dispensing can be activated by squeezing the flexible body portion with the user's finger pressure to force the fluid product from the body portion and into and through the needle or luer that has punctured the membrane, and out the other open end of the needle or luer for delivery to a person or animal.

14. The stirp of claim 1, wherein the single plastic material is made of a single recyclable material.

15. The method of claim 6, wherein
the filling is done at room temperature, allowing the filling of heat-sensitive products.
